(19) United States
(12) Patent Application Publication
(10) Pub. No.: US 2012/0006101 A1
(43) Pub. Date: Jan. 12, 2012

(54) Method for determining the optimal treatment dosage for metals removal

(76) Inventor: David Allen Wensloff, (US)

(21) Appl. No.: 12/830,617

(22) Filed: Jul. 6, 2010

(51) Int. Cl. G01N 33/20

(52) U.S. Cl. 73/61.42

(57) ABSTRACT

Wastewater from industrial processes commonly contains dissolved metals which require removal in order to meet discharge limits. The metal precipitant IE 061 may be utilized along with Floccin Agents™ for the removal of dissolved metals. The method here described indicates how to determine the appropriate dosage of IE061 metal precipitant and Floccin Agents™ required to treat the water. The method utilizes pH adjustment to an insoluble level for metals of concern. IE061 is then incrementally added to the water until the metals have precipitated utilizing oxidative state as an indicator of level of soluble metals. Required concentrations are determined by adding precipitant until the oxidative state indicates that no further precipitation is achievable. A Floccin Agent™ is then added in measured incremental steps in order to coagulate, flocculate and encapsulate the insoluble metals. The Floccin Agent™ is added incrementally to determine the concentration that yields acceptable metals removal.

### REFERENCES CITED

1. Langner; Herbert G. J. (Burlington, Ontario, CA)
2. Grunenwald; Edward D. (Boalsburg, Pa.)

### U.S. Patent Documents

| | | |
|---|---|---|
| 5,505,857 | April 1996 | Misra, et al |
| 3,931,007 | Jan 1976 | Sugano, et al.. |
| 4,943,377 | July 1990 | Legare, III |
| 4,758,353 | July, 1988 | Spence, et al. |
| 3,932,274 | January 1976 | Izumi et al. |

### BACKGROUND OF THE INVENTION

The current process relates to the field of wastewater treatment and more specifically to the removal of metals from industrial wastewater. There are many industrial processes which produce water that contains one or more dissolved metals. Because the metals are dissolved, they are generally visually undetectable. Nevertheless, water with dissolved metals is toxic to humans and can pose a danger. It is therefore necessary to remove the metals from solution and bring them out of the water.

Industrial processes which produce metal laden water are often required to utilize wastewater treatment systems in order to remove unwanted contaminants from the waste stream. These systems often combine a mechanical and chemical treatment in the process of removing constituents of concern from the water. The variety of wastewaters produced by industry result in numerous techniques utilized to treat water.

Many facilities such as plating operations utilize chelating agents such as EDTA to keep metals in solution thereby insuring a uniform and continuous layer of plated metal. Often such chelating agents continue to bind metal ions in a soluble state even when the pH is adjusted to the proper insolubility point to form metal hydroxides. This means pH adjustment along with coagulation and flocculation is insufficient to meet compliance standards. To reduce these chelated metals they need to be precipitated. The metal precipitant, IE061 is utilized to bring the metals out of solution as necessary in many cases to be in compliance with government regulation.

In order to cost effectively treat water laden with soluble metals it is important to determine the required quantities of precipitant and flocculent to achieve the desired level of treatment. The effectiveness of waste water treatment operations is hindered by the excess application of treatment chemicals.

It is common to perform jar tests in order to determine the type and quantity of chemical required for effective water treatment. A representative sample of the water to be treated is placed in a beaker and treated on a small scale. This allows for the quantification treatment chemicals on a small scale. The concentration of chemicals required for treatment as determined by the jar test may then be applied to wastewater on a larger scale.

The need exists for a process to determine the appropriate dosage of the metal precipitant IE061 utilizing the ORP control in combination with the Floccin™ line of flocculating agents. The process utilizes the scaled down treatment in with jar testing along with procedures for determining the most effective quantities of IE061 metal precipitant and Floccin™ Agents for a given waste water sample.

### SUMMARY OF THE INVENTION

The present invention is a process which determines the optimal amount of metal precipitant and Floccin™ Agent to add to a waste stream for the removal of metals. The process utilizes the addition of IE 061, a calcium polysulfide based metal precipitant and Floccin agents which are used to aid coagulation and flocculation in wastewater treatment. A sample of wastewater is contained in a 1000 ml beaker and the pH is adjusted to a level determined by the known concentration of metals of concern in the water. The known pH levels for insolubility are utilized to determine the best pH for insolubility of the metals in wastewater and the pH is adjusted accordingly.

Once the pH has been adjusted, the metal precipitant, IE061 is added. The IE061 is added incrementally until no more metals come out of solution. The Oxidation Reduction Potential (ORP) is an indicator for the quantity of metal in solution. The ORP value will therefore change along with a change of the quantity of metals in solution. Consequently, when the addition of IE061 fails to yield a change in the ORP value, it may be concluded that the quantity of metal in solution is not changing and the addition of IE061 is no longer necessary.

After the metal is precipitated, Floccin is added incrementally while the water is mixed continuously. The wastewater is allowed to mix for a period of time after each increment of Floccin is added so that it can be observed to determine if flocculation has occurred. The incremental addition of Floccin is continued, followed by observation until the acceptable flocculation has occurred at which point the concentration of chemicals added to the water may be recorded.

### DESCRIPTION OF PREFERRED EMBODIMENTS

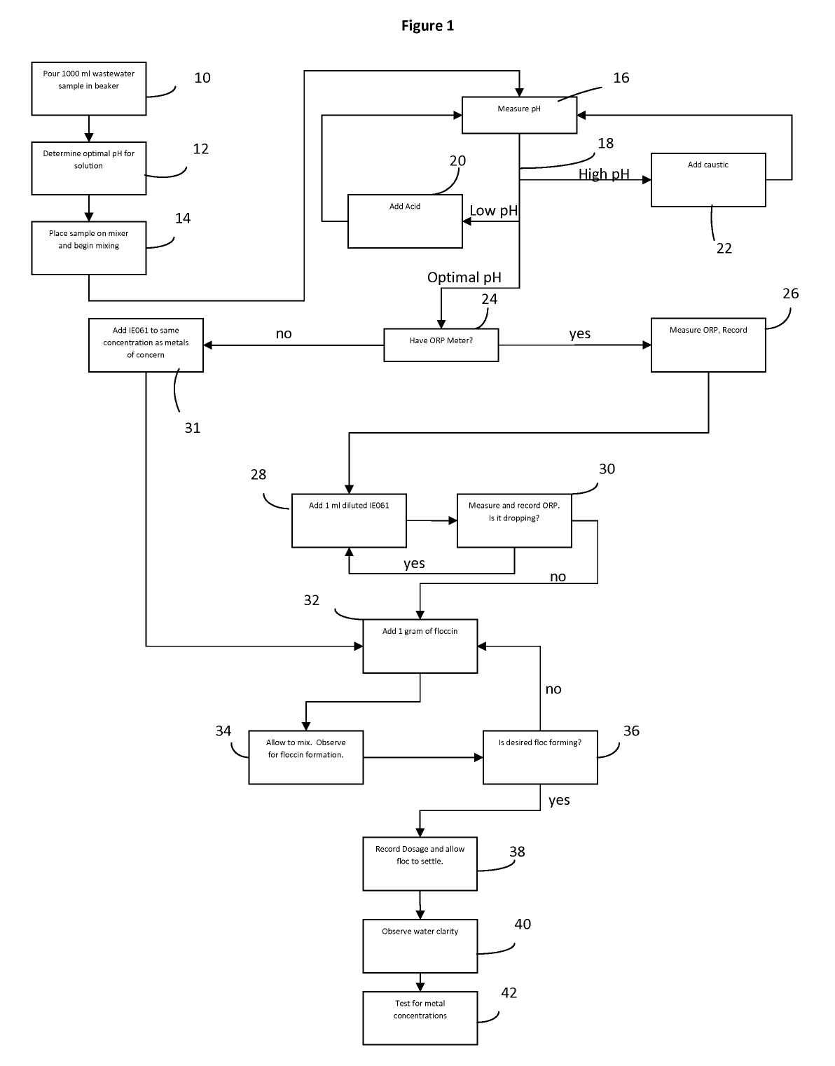

FIG. 1 is a flow chart which indicates the standard procedure for implementing the this process.

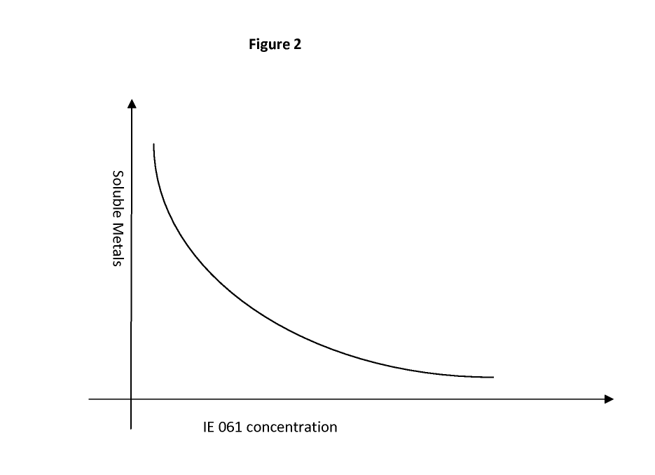

FIG. 2 is a chart which indicates the relationship between the concentration of IE061 and th soluble metals in the wastewater.

IE-061 and Floccin agents are used to precipitate and remove metals from waste water. A process is utilized to determine the required quantity of IE-061 and Floccin agent to effectively treat a waste stream. This process utilizes common tools in the realm of waste water treatment in order to establish the most cost effective quantity of the metal precipitant IE-061 and the one coagulating and flocculating agents referred to as Floccin.

In order to determine the optimal IE061 dosage and Floccin dosage for a given waste stream, a sample of the water should be obtained. The water should be poured into a 1000 ml beaker (10) and the optimal pH should be determined for the water base upon know values for insolubility of metals as shown in Table 1 (12).

TABLE 1

| Metal | Insoluble pH |
|---|---|
| Aluminum (Al) | 10 |
| Cadmium (Cd) | 11 |
| Chrome (Cr) | 9.0 |
| Copper (Cu) | 8.9 |
| Lead (Pb) | 9.5 |
| Nickel (Ni) | 10.2 |
| Zinc (Zn) | 8.8 |

Typically facilities have two, three or more different metals in their waste stream. To determine the optimal pH, use a concentration factor of each metal and calculate the optimal pH point. The following equation should be used to determine the optimal pH for insolubility. For x number of metals the optimal pH can be found with the equation below.

$$Optimal pH = \frac{{C}_{1} {pH}_{1} + {C}_{2} {pH}_{2} … + {C}_{x} {pH}_{x}}{{C}_{1} + {C}_{2} … + {C}_{x}}$$

Where C₁ is the concentration of metal 1, pH is the insoluble pH value for metal 1.

Once the optimal pH has been determined the pH of the solution should be adjusted to the optimal pH value. The 1000 ml beaker of water should be placed on a mixer (14) which continuously stirs the solution to ensure proper mixing while the pH is adjusted. The pH should be adjusted slowly to avoid overshooting the desired pH value. This is accomplished by the incremental addition of caustic or acid while continuously measuring the pH with a pH probe until the desired pH is reached. The pH should be measured (16), and if the pH is high, acid should be added (20) if the pH is low, caustic should be added (22). The pH measurement and adjustment should be repeated until the optimal pH is reached.

Once the optimal pH has been attained, a dilute solution of IE 061 and distilled water should be made. The IE 061 should be diluted to 10% in solution. This is done for ease of measurement as 1 ml 10% IE 061 in 100 ml of water is equivalent to 100 ppm. An ORP meter should be utilized to determine the required dosage for insolubility if one is available (24).

The ORP meter measures the Oxidation Reduction Potential which changes in relation to the dissolve metals in the wastewater. The measure of the oxidative state of is an indication of the amount of metals that are soluble. The chart in FIG. 2 below the general relationship between the ORP value, as shown on the horizontal axis, and the insoluble metals in the water, as shown on the vertical axis.

As more precipitant is added, the metals come out of solution and the ORP reading drops. As more precipitant is added, the graph starts to level off until adding more precipitant causes no significant decrease of the metals in solution and correspondingly, no significant change in the measured ORP value.

The relationship between the oxidative state and the soluble metals can be used to determine when the precipitant has reached its effective limit. The ORP should be measured (26) followed by the addition of 1 ml of IE061 diluted to 10% (28) which is equal to 100 ppm in a 1000 ml beaker. The ORP should then be measured and the value should be noted (30). The diluted IE 061 should be added in 1 ml increments until and the ORP measured until no significant change in the ORP value is observed. At this point additional precipitant will have no measurable effect on the level of soluble metals. The concentration of IE 061 should be noted as the concentration required to bring metals out of solution.

Alternatively, if no ORP meter is available, IE061 should be added to a concentration equal to the concentration of the metals in the water (31). This will precipitate the metals from the water in order to proceed with the next step. The use of an ORP meter is preferable to this method.

Once enough IE061 has been added, Floccin should be added incrementally to determine how much is required for a good floc to form. This is accomplished by adding floc a gram at a time, allowing it to mix and observing the solution to determine if an acceptable floc has formed. After the first gram is added (32), the solution should be allowed to mix for several minutes and observed to determine if acceptable flocculation has occurred (36). If an acceptable floc has formed, the mixer may be turned off and the floc allowed to settle (38). If not, another gram of Floccin should be added and allowed to mix (32). Floccin should be added a gram at a time until an acceptable floc is observed in the water. Once an acceptable floc has formed, it should be allowed to settle and the water clarity should be observed (40). The concentration should be recorded as the optimal Floccin concentration for the wastewater sample. The solids can then be removed and the treated water can be tested for metals of concern (42).

### DESCRIPTION OF RELATED ART

The necessity of removal of metals and solids from water has given rise to number of techniques and operations. The need to remove metals from solution in water is generally recognized and many methods have been used to effectively achieve this end. There remains a need when utilizing IE061 metal precipitant and Floccin agents for an effective method of determining the dosage of IE 0061 and Floccin agent that will be effective for treatment of a given wastewater sample.

U.S. Pat. No. 5,505,857 describes a process in which inorganic sulfur compounds are used in conjunction with pH adjustment to precipitate metal from a solution in water. This process illustrates a method for the treatment of water with soluble metals.

U.S. Pat. No. 3,931,007 describes a method for the removal of heavy metals in water that utilizes pH adjustment, the addition of a metal precipitant and the encapsulation of impurities for removal from the water. This process describes a full scale treatment of water to meet the needs of wastewater with soluble metals.

U.S. Pat. No. 4,943,377 describes a method in which precipitation of metals is achieved by mixing a solution of sodium polythiocarbonate with the solution containing the heavy metals. The precipitated metals are then removed through gravity or gravity assisted settling.

U.S. Pat. No. 4,758,353 describes a method of removing heavy metals from a waste stream by means of surfactant addition, pH adjustment, adding coagulant and then flocculant. This method assumes an appropriate concentration of chemicals for effective treatment. This method differs from others described as it does not employ a metal precipitant to remove metals from solution.

A metal Sequestering agent is described in U.S. Pat. No. 3,932,274 for capturing metal ions in water. Sequestering metals in solution is not uncommon in many industries and may hinder the solubility of metals due to pH adjustment.

The related patents illustrate the general need for a method of treating water with soluble metals. They also demonstrate the need for precipitating agents in the process of removing metals from solution.

The use of Jar Testing in wastewater is a well know method for determining the Many methods and techniques have been developed for the removal of solids from wastewater. The present process is designed to determine the appropriate combination of IE061 metal precipitant and

Floccin product in order to remove metals from wastewater. The present process is utilized as a means of determining what the appropriate dosage is for the removal of metals in a waste stream.

IE061 and Floccin agents are proprietary chemicals utilized for the treatment of a variety of waste water streams.

1. A process for the determination of the optimal concentration of metal precipitant and flocculation agents which comprises:
a. Adjustment of wastewater pH to the optimal level for the precipitation of known metals
b. The addition of metal precipitant IE061
c. The addition of Floccin Agents™ to the wastewater

2. The process of claim 1 wherein an ORP meter is used to:
a. measure the oxidation reduction potential of the wastewater solution
b. determine the point at which additional precipitant fails to yield a change in soluble metals.

3. A process of claim 1 wherein the Floccin Agents™ are added incrementally to determine the concentration that yields acceptable flocculation of the wastewater.

4. A process of claim 3 wherein the water is clarified forming clear treated water and a sludge which may be dewatered.

5. A process of claim 4 wherein the treated water is tested for concentrations of metals of concern.

6. The Process of claim 3 wherein the dewatered sludge is tested utilizing the US EPA TCLP and STLC testing protocols to determine the leachability of the sludge.
